(19) United States
(12) Patent Application Publication
(10) Pub. No.: US 2016/0011168 A1
(43) Pub. Date: Jan. 14, 2016

(54) METHOD FOR JUDGING PRESENCE OR ABSENCE OF EPITHELIAL-MESENCHYMAL TRANSITION

(71) Applicant: SHIMADZU CORPORATION, Kyoto-shi (JP)

(72) Inventors: Noriko IWAMOTO, Kyoto-shi (JP); Takashi SHIMADA, Kyoto-shi (JP)

(73) Assignee: SHIMADZU CORPORATION, Kyoto-shi (JP)

(21) Appl. No.: 14/794,505

(22) Filed: Jul. 8, 2015

(30) Foreign Application Priority Data
Jul. 8, 2014 (JP) 2014-140544

(51) Int. Cl. G01N 33/483

(52) U.S. Cl. CPC G01N 33/4833; G01N 2800/52

(57) ABSTRACT

A method for detecting epithelial-mesenchymal transition of a cell includes measuring a level of serine in a metabolic substance mixture prepared from culture of the cell, and comparing the level of serine with a reference for serine such that epithelial-mesenchymal transition of the cell is detected based on at least the level of serine compared with the reference.

### CROSS-REFERENCE TO RELATED APPLICATIONS

The present application is based upon and claims the benefit of priority to Japanese Patent Application No. 2014-140544, filed Jul. 8, 2014, the entire contents of which are incorporated herein by reference.

### BACKGROUND OF THE INVENTION

1. Field of the Invention

The present invention relates to metabolite variation that is associated with epithelial-mesenchymal transition and relates to a method for judging presence or absence of the epithelial-mesenchymal transition.

2. Description of Background Art

Epithelial-mesenchymal transition (EMT) plays a central role in malignant progression of cancer and is also involved in cancer metastasis (Non-Patent Literature 1). On the other hand, it has been so far difficult to temporally and spatially track epithelial-mesenchymal transition in a tumor.
Non-Patent Literature 1: Nat Rev Cancer 2002 Jun.; 2(6): 442-54.
Non-Patent Literature 2: Mani S et al (2008) Cell, 133(4): 704-715.
Non-Patent Literature 3: Cancer Science 2013 March; 104(3): 398-408.

### SUMMARY OF THE INVENTION

According to one aspect of the present invention, a method for detecting epithelial-mesenchymal transition of a cell includes measuring a level of serine in a metabolic substance mixture prepared from culture of the cell, and comparing the level of serine with a reference for serine such that epithelial-mesenchymal transition of the cell is detected based on at least the level of serine compared with the reference.

According to another aspect of the present invention, a method for detecting epithelial-mesenchymal transition of a cell includes measuring a level of serine in a metabolic substance mixture prepared from a tissue sample, and comparing the level of serine with a reference for serine such that epithelial-mesenchymal transition of the cell is detected based on at least the level of serine compared with the reference.

According to still another aspect of the present invention, a method for detecting epithelial-mesenchymal transition of a cell includes measuring a level of serine in a metabolic substance mixture prepared from culture of a cell taken from a subject, and comparing the level of serine with a previous level of serine measured for the subject such that epithelial-mesenchymal transition of the cell is detected based on at least the level of serine compared with the previous level of serine.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 illustrates forms of HepG2 cells treated with BMP-9. Protruding structures that are morphological features of cells that have undergone epithelial-mesenchymal transition are observed and transparent cells have occurred. It is confirmed that, due to the BMP-9 treatment, EMT time-dependently progresses in the HepG2 cells.

FIG. 2 illustrates alkaline phosphatase activity in the HepG2 cells that are treated with BMP-9. Increase in the alkaline phosphatase activity is a feature of cancer stem cells.

### DETAILED DESCRIPTION OF THE EMBODIMENTS

The embodiments will now be described with reference to the accompanying drawings, wherein like reference numerals designate corresponding or identical elements throughout the various drawings.

In some embodiments, the present invention provides an analysis method for determining whether or not cells have undergone epithelial-mesenchymal transition. The method includes i) a step of measuring metabolites such as amino acids (such as serine, aspartic acid, phenylalanine) that are extracted from cells that are suspected to be cells that have undergone epithelial-mesenchymal transition, or from culture of the cells, and ii) a step of comparing the measurement result with a reference. The cells may be cancer cells.

Further, in some embodiments, the present invention provides a method for analyzing whether or not cells that have undergone epithelial-mesenchymal transition are detected from a tissue. The method includes i) a step of measuring metabolites such as amino acids (such as serine, aspartic acid, phenylalanine) that are extracted from a tissue that is suspected to include cells that have undergone epithelial-mesenchymal transition, and ii) a step of comparing the measurement result with a reference. The cells may be cancer cells.

In the present specification, a reference for a certain metabolic substance refers to a metabolic substance level in cells that have not undergone epithelial-mesenchymal transition and that correspond to cells to be analyzed that have undergone epithelial-mesenchymal transition. The metabolic substance level (standard level) in the cells that have not undergone epithelial-mesenchymal transition can be established by suitable experiments and statistical analysis. The standard level may be an average or median value of a relative amount or an absolute amount. A difference between a measured value and the standard level, a threshold and a cut-off value can be arbitrarily set according to intended sensitivity and specificity.

As a result of comparing with a reference, when levels of serine and/or aspartic acid in a sample is lower than the reference or a level of phenylalanine in the sample is higher than the reference, it can be determined that test cells are cells that have undergone epithelial-mesenchymal transition, or it can be determined that cells that have undergone epithelial-mesenchymal transition are contained in a test tissue. The cells may be cancer cells. The comparison includes not only comparing, for example, a measured metabolic substance level with a reference level, but also examining a variation rate.

In the present specification, when it is referred that it is “determined” that the test cells are cells that have undergone epithelial-mesenchymal transition, it is preferable that the determination is 100% correct. However, this may be not the case in practice. However, in practice, it is sufficient to be able to “determine” with a certain sensitivity and specificity that the test cells are cells that have undergone epithelial-mesenchymal transition. Or, depending on a purpose, it is sufficient to be able to determine that a probability that the test cells are cells that have undergone epithelial-mesenchymal transition is high. The same also applies to “detection.” In the present specification, when it is referred that cells that have undergone epithelial-mesenchymal transition are “detected” from a test tissue, it is preferable that the detection is 100% correct. However, this may be not the case in practice. However, in practice, it is sufficient to be able to “detect” with a certain sensitivity and specificity cells that have undergone epithelial-mesenchymal transition from a tissue. For a person of ordinary skill in the art, what reference value should be adopted in order to satisfy intended sensitivity and specificity can be derived using various statistical evaluation methods. Examples of the statistical methods include determination of confidence intervals, determination of p values, student's t-test, and the like.

Regarding a detection method in the present specification, that the sensitivity of detecting cells that have undergone EMT from a sample is high means that the detection can be performed even when the number of the cells that have undergone EMT in the sample is small. Further, in the present specification, regarding the detection of the cells that have undergone EMT, that the specificity is high means that the probability of correctly determining negative specimens as being negative in an examination is high. The sensitivity and specificity can be arbitrarily set according to a purpose of an examination.

Metabolism variations of amino acids such as serine, aspartic acid and phenylalanine are each an indicator of whether or not cells have undergone epithelial-mesenchymal transition. The metabolic substance to be measured is not limited to one type. Two, three, four or more types of metabolic substances can be suitably combined. That is, for a sample, not only the metabolism variation of serine is measured, but further the metabolism variations of aspartic acid and phenylalanine can also be measured. In this case, as a matter of course, the references respectively correspond to the metabolic substances. That is, when serine is measured, the result is compared with the reference for serine; when aspartic acid is measured, the result is compared with the reference for aspartic acid; and when phenylalanine is measured, the result is compared with the reference for phenylalanine.

Examples of the metabolic substances as indicators for whether or not cells have undergone epithelial-mesenchymal transition include the following. That is, when intracellular metabolic substance variation is analyzed, examples of the metabolic substances as the indicators include, but are not limited to, isoleucine, valine, leucine, alloisoleucine, proline, threonine, 4-hydroxyproline, cysteine, glutamine, d-glucose, tyrosine, palmitoleic acid, oleic acid, stearic acid, 3-D-mannopyranoside, glycine, acetylaspartic acid, D-(−)-tagatose, and palmitic acid. Further, when extracellular metabolic substance variation is analyzed, examples of the metabolic substances as the indicators include, but are not limited to, fumaric acid, amino malonic acid, malic acid, methionine, asparagine, citric acid, glycerol, L-proline, tryptophan and cholesterol. Also for these metabolic substances, as a matter of course, the references respectively correspond to the metabolic substances. These indicators can each be used alone or in combination with other indicators. For example, one or more of these indicators can be measured in combination with one or more of serine, aspartic acid and phenylalanine. Further, other indicators or markers may be further measured.

More specifically, when intracellular metabolic substance variation is analyzed, by comparing with the references, increases in valine, leucine, alloisoleucine, proline, glycine, threonine, 4-hydroxyproline, cysteine and tyrosine are indicators that the cells have undergone epithelial-mesenchymal transition; and decreases in isoleucine, acetylaspartic acid, glutamine, D-(−)-tagatose, d-glucose and β-D-mannopyranoside are indicators that the cells have undergone epithelial-mesenchymal transition. Further, when extracellular metabolic substance variation is analyzed, by comparing with the references, increases in glycerol, L-proline, fumaric acid, citric acid and malic acid are indicators that the cells have undergone epithelial-mesenchymal transition; and decreases in amino malonic acid, asparagine, tryptophan, methionine and cholesterol are indicators that the cells have undergone epithelial-mesenchymal transition.

By analyzing metabolic conversion of the cells and further analyzing various genetic markers and gene expression patterns with respect to the cells that are determined to be cells that have undergone epithelial-mesenchymal transition, whether or not the cells that have undergone epithelial-mesenchymal transition are cells having metastatic potential and further whether or not the cells have properties of cancer stem cells can be analyzed. Examples of the genetic markers for analyzing whether or not the cancer cells have metastatic potential include, but are not limited to, E-cadherin. Examples of the genetic markers for analyzing whether or not the cells have properties of cancer stem cells include, but are not limited to, CD19, CD20, CD24 (HSA), CD38, CD44 (PGP1), CD90 (THY1), CD133 (prominin 1), EpCAM (epithelial cell adhesion molecule), E-cadherin and ATP-binding cassette B5 (ABCB5). Information about the genetic markers can be obtained from a known database such as GenBank.

The methods described in the present specification may be an in vitro method that is performed in vitro. By determining whether or not the cancer cells are cells that have undergone epithelial-mesenchymal transition, it is possible to determined prognosis of the cancer, to predict presence or absence of metastatic potential, and to evaluate an effect of cancer treatment. The analysis according to the methods described in the present specification includes monitoring the cells that have undergone epithelial-mesenchymal transition and confirming whether or not the cells that have undergone epithelial-mesenchymal transition exist even after treatment with the drug candidate compound. The monitoring means, for example, examining presence or absence of the cells that have undergone epithelial-mesenchymal transition during a cancer treatment period or measuring the number of the cells. Further, the methods described in the present specification can also enhance or reinforce diagnosis using other methods. The enhancing or reinforcing means reinforcing or validating identification or determination that is performed using indicators and genetic markers other than metabolism variations.

In some embodiments, the present invention determines whether or not cancer cells are cancer cells that have undergone epithelial-mesenchymal transition or whether or not cancer cells that have undergone epithelial-mesenchymal transition are contained in a tissue. Therefore, the methods according to some embodiments of the present invention can be performed using arbitrary cancer cells or cancer tissue samples. Examples of the cancer cells include cancer cells of liver cancer, renal cell cancer, lung cancer, stomach cancer, large intestine cancer, colon cancer, small intestine cancer, pancreas cancer, spleen cancer, bladder cancer, prostate cancer, testicular cancer, uterine cancer, ovarian cancer, breast cancer, lymphoma, bone marrow cancer, brain tumor, neuroblastoma, tongue cancer, pharyngeal cancer, esophagus cancer, thyroid cancer, parathyroid cancer, sarcoma, myeloma, bone sarcoma, glandular cancer, skin sarcoma, metastatic cancer, and the like. Examples of the tissues include cancer tissues of liver, kidney, lung, stomach, large intestine, colon, small intestine, pancreas, spleen, bladder, prostate, testis, uterus, ovary, breast, skin, blood vessels, blood, bone marrow, brain, nerve, tongue, pharynx, esophagus, muscle, skeletal muscle, skeletal, endocrine, sarcoma, osteosarcoma, tumors, malignant tumors, and the like. Subjects from which samples are collected include animals such as mammals such as mouse, rat, monkey, human, and the like.

The samples may be cell samples, tissue samples and medium samples. When a sample is cells, when the amount of the cells is small, the cells may be cultured in advance and preparation and analysis of metabolic substances may be performed with respect to the cultured cells. When a sample is a tissue, a metabolic substance can be prepared from the tissue or cancer cells can be obtained from the tissue and subjected to analysis after culture. When analysis is performed using GC-MS, LC-MS or the like to be described below, although it also depends on a device that is used, a cell sample includes, for example, 10⁵ or more, preferably, 10⁶ or more cells. When the number of the cells in the sample is less than this, the obtained cells can be preliminarily cultured and then subjected to analysis. When a sample is a medium that contains a metabolic substance that is extracellularly secreted, a medium sample of 10 μL or more, for example, 100 μL or more can be used. The sample may be collected by a suitable means such as tissue biopsy, needle biopsy or the like.

A metabolic substance mixture (may be referred to as a metabolome) is prepared from the obtained sample. The prepared metabolic substance mixture may be an intracellular metabolic substance, an extracellular metabolic substance, and a mixture of these. Depending on a purpose, it is possible to analyze only an intracellular metabolic substance, to analyze only an extracellular metabolic substance, to separately analyze the intracellular metabolic substance and the extracellular metabolic substance, or to analyze a mixture of both.

The preparation of the metabolic substance mixture may include suitable steps such as cell culturing, cleaning, ultrasonic fragmentation, centrifugal separation, extraction, fractionation, concentration and purification. In some embodiments, when gas chromatography (GC) is used as a measurement method, the metabolic substance is derivatized in advance. The derivatization includes, for example, methoxylation and trimethylsilylation of a polar compound, and transmethylation, methoxylation and trimethylsilylation of a non-polar compound.

A metabolic substance contained in a sample can be quantitatively or qualitatively measured. When a metabolic substance is amino acid, sugar, fatty acid, or a small molecule compound, the measurement is preferably quantitative measurement (quantitative analysis).

Measurements of metabolic substances can be performed using suitable methods. Examples of the measurement methods include all chromatographic separation methods such as gas chromatography (GC), liquid chromatography (LC), high performance liquid chromatography (HPLC), thin layer chromatography (TLC), affinity chromatography and the like. The examples further also include mass spectrometry (MS), capillary electrophoresis analysis (CE), two-dimensional electrophoresis, NMR analysis, and the like.

The mass spectrometry used in the present specification includes all technologies that enable measurement of molecular weights (that is, masses) or mass changes of corresponding metabolic substances. A mass spectrometer includes a sample introduction part, an ionization chamber, an analyzer, a detector, a recorder and the like. As an ionization method, a chemical ionization method, a field desorption (FD) method, a fast atom bombardment (FAB) method, a matrix-assisted laser desorption ionization (MALDI) method, an electrospray ionization (ESI) method, or the like can be used. As the analyzer, a double-focusing mass spectrometer, a quadrupole mass spectrometer, a time-of-flight mass spectrometer, a Fourier transform ion cyclotron resonance mass spectrometer, or the like can be used.

The measurement methods may each be used alone or a plurality of the measurement methods may be combined. Therefore, the analysis includes combinations of various chromatographic separation methods and various types of mass spectrometry such as gas chromatography-mass spectrometry (GC-MS), liquid chromatography-mass spectrometry (LC-MS), Fourier transform ion cyclotron resonance mass spectrometry (FT-ICR-MS), capillary electrophoresis analysis-mass spectrometry (CE-MS), high-performance liquid chromatography-mass spectrometry (HPLC-MS), inductively-coupled plasma-mass spectrometry (ICP-MS), and pyrolysis mass spectrometry (Py-MS), and further includes MS-MS and combinations of these. Preferably, the analysis of the metabolic substances is performed using the liquid chromatography-mass spectrometry (LC-MS) or the gas chromatography mass spectrometry (GCMS). In the GC/MS analysis, total ion chromatogram (TIC) scan and/or selective ion monitoring (SIM) analysis can be performed. When a mixture of a large number of metabolic substances (metabolome) is analyzed, the metabolic substances can each be individually identified by using a scan mode. The metabolome means an entire set of small molecule compounds that are contained in a biological sample. Thereafter, each identified individual metabolic substance can be quantitatively analyzed with high accuracy in an SIM mode by specifying the mass of the metabolic substance.

The present inventors analyzed a metabolic substance mixture of cells that have undergone epithelial-mesenchymal transition using liver cancer cells HepG2 as a model and, as a result, found that significant metabolism variations are observed with respect to amino acids such as serine, aspartic acid and phenylalanine. Based on this finding, references are also similarly established for all other types of cancer cells by measuring metabolism variations of amino acids such as serine, aspartic acid and phenylalanine of cancer cells and cancer cells that have undergone epithelial-mesenchymal transition. Based on this, whether or not cells have undergone epithelial-mesenchymal transition can be determined. The cancer cells for establishing such references can be obtained by causing cancer cells that have not undergone epithelial-mesenchymal transition to undergo EMT. EMT can be caused to occur by processing cancer cells in presence of a substance that can induce EMT such as bone morphogenetic protein-9 (BMP-9) in the case of liver cancer cells.

Using the findings about the metabolism variations in the epithelial-mesenchymal transition cells as described in the present specification, drug candidate compounds can be screened. For example, when a method according to one aspect of the present invention is applied to a tissue sample after administration of a drug and epithelial-mesenchymal transition cells are not detected, it indicates effectiveness of the drug.

In a screening method according to one aspect of the present invention, first, cancer cells and a drug candidate compound are put in contact with each other and are cultured. Next, with respect to the culture, whether or not growth of the cancer cells is inhibited or the number of the cancer cells is reduced is observed. When growth inhibition in the cancer cells or reduction in the number of the cells is observed, with respect to the cells, a metabolic substance mixture is prepared from the cell culture. With respect to the metabolic substance mixture, one or more amino acids selected from the group consisting of serine, aspartic acid and phenylalanine are measured. The measurement result is compared with the reference corresponding to the metabolic substance. When cells that have undergone epithelial-mesenchymal transition are not detected from the cell culture by comparing with the reference, it can be determined that the drug candidate compound affects the cancer cells that have undergone epithelial-mesenchymal transition.

Examples of the drug candidate compounds include, but are not limited to, low molecular compounds, peptide, polypeptide, protein, transcription factors, antibodies, nucleic acids and the like. Compounds that are contained in suitable compound libraries and combinatorial libraries may be used in the screening.

The metabolism variation measurement method according to one aspect of the present invention can detect cells that have undergone epithelial-mesenchymal transition. This can be used to help determine whether a cancer tissue is from a primary tumor or from a metastatic tumor. The epithelial-mesenchymal transition (EMT) is believed to be involved in cancer metastasis. Therefore, for example, when cells that have undergone the epithelial-mesenchymal transition are detected from a cancer tissue that is surgically excised from a tumor, it can be determined that the probability that the tumor is a metastatic tumor is high, and otherwise it can be determined that the probability that the tumor is a primary tumor is high.

The metabolism variation measurement method according to one aspect of the present invention can be used to detect cells that have undergone epithelial-mesenchymal transition, and can also be used as an auxiliary measurement means. In some embodiments, the method according to one aspect of the present invention can be helpful in companion diagnosis. The companion diagnosis or companion examination refers to examination that is performed before administration of a drug in order to predict presence or absence of effects or side effects of the drug. Companion diagnosis can
identify patients who are most likely to benefit from a particular therapeutic product;
identify patients likely to be at increased risk for serious side effects as a result of treatment with a particular therapeutic product; or
monitor response to treatment with a particular therapeutic product for the purpose of adjusting treatment to achieve improved safety or effectiveness.
   As a result, reactions of an individual patient to the drug can be grasped before treatment, which can help in dose adjustment and in treatment regimen design.

### Example

Next, the present invention is described with reference to an example. The technical scope of the present invention is not limited by the following example.

Preparation of Cells that have Undergone EMT

Established cell lines (HepG2) of liver cancer cells were used as the cells (obtained from ATCC; ATCC registration number HB-8065). A medium obtained by adding an antibiotic substance, 10% of FBS, and L-alanylglutamine to a DMEM medium was used as the medium. EMT was induced by stimulating liver cancer cells (HepG2) with EMT inducing cytokine BMP-9 (R&D Systems) (Non-Patent Literature 3, Li Q et al Cancer Sci. 2013). Conditions for the cells to undergo EMT were to add 50 ng/mL of BMP-9 to the medium and to perform culture for three days. EMT of the HepG2 cells was confirmed using a wound healing method and by alkaline phosphatase activity measurement.

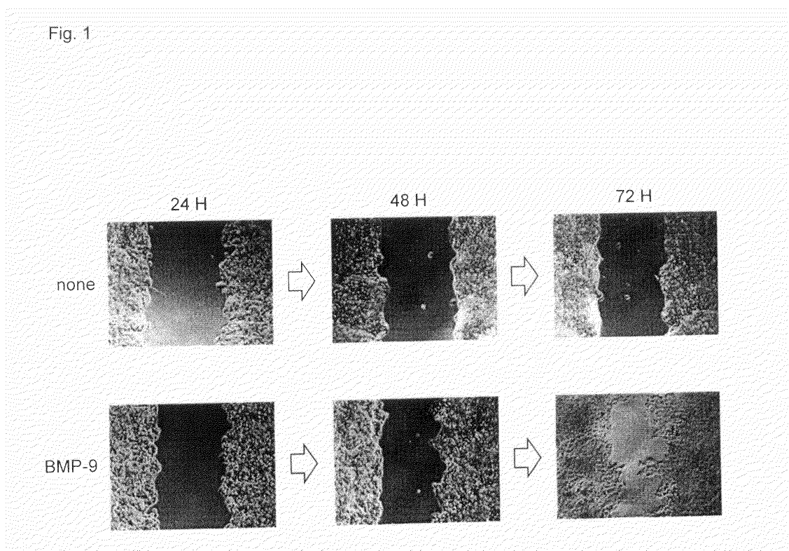

Specifically, BMP-9 (50 ng/mL) was caused to react with the HepG2 cells and then the HepG2 cells were placed in a CO₂ incubator for 24, 48 and 72 hours, and EMT of the HepG2 cells was confirmed using the wound healing method. As illustrated in FIG. 1, the HepG2 cells of the liver cancer cells morphologically changed due to the cytokine BMP-9 treatment, and changed to cells that have undergone EMT in a reaction time-dependent manner.

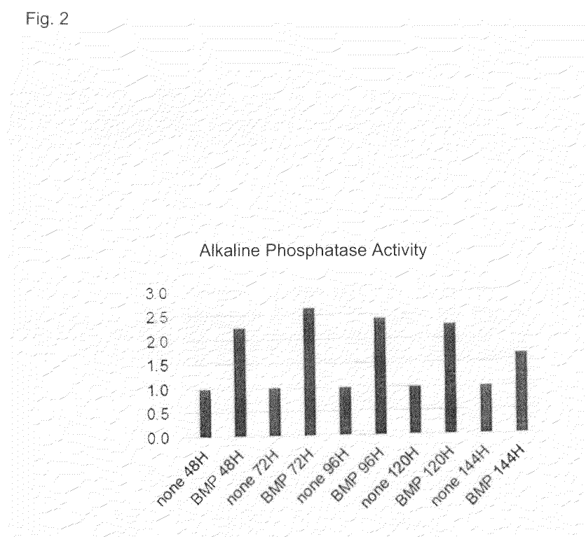

Further, BMP-9 (50 ng/mL) was caused to react with the HepG2 cells and then the HepG2 cells were placed in a CO₂ incubator for 24, 48, 72, 96, 120 and 144 hours, and alkaline phosphatase activity was measured. As illustrated in FIG. 2, the alkaline phosphatase activity, which is an indicator of properties of stem cells, was most active 72 hours after the BMP-9 stimulation. As described above, from FIGS. 1 and 2, that the EMT of the HepG2 cells of the liver cancer cells was confirmed.

Preparation of Metabolites

Next, metabolites of cells that have undergone EMT were prepared by the following steps. Specifically, intracellular metabolites were extracted by collecting culture cells after washing and removing the medium with PBS (the number of the cells was 4×106), adding 80% of MeOH (−80° C.) to perform ultrasonic treatment, and collecting centrifuged supernatant. This operation was repeated three times with respect to the same sample. Metabolites in an extracellularly secreted medium were extracted by adding MeOH (−80° C.) (400 μL) to the medium (100 μL) and vigorously mixing with a vortex for 10 minutes, and collecting supernatant after centrifugation at 16,000 g for 10 minutes. As an internal standard substance in each of the extracted metabolites, 2-isopropylmalic acid (1 μg) was added to each of the samples, which were then centrifuged and dried using a SpeedVac and were stored at −80° C. until measurement was performed.

Measurement Method of Metabolites Using GCMS

The measurement of the metabolites uses GCMS. Therefore, trimethylsilyl derivatization of the metabolite was performed. Methoxyamine hydrochloride (20 μL) of 20 μg/μL (dissolved in pyridine) was added to a sample. The sample was well dispersed by ultrasound and was incubated for 90 minutes under a condition of 37° C. while being shaken at 12,000 rpm. Next, 60 μL of MSTFA (GL Science) was added to the sample and sample was incubated for 180 minutes at 37° C. while being shaken at 12,000 rpm. After completion of the reaction, the sample was centrifuged for 10 min at 16,000 g, and the supernatant was used as a sample for the GCMS measurement.

A GCMS-TQ8030 (Shimadzu Corporation) was used for the measurement. An Rxi-5Sil MS (30 m)×(0.25 mm) ID df. 0.25 μM (RESTEK) was used for a capillary column for GC. Temperature of an inlet was 280° C., and a helium gas was used as a carrier gas and was flowed at 39.0 cm/sec (constant linear speed mode). A temperature raising condition of a GC column was 100° C. (2 min)-4° C./min-320° C. (10 min). The analysis was performed at electron ionization energy of 0 eV, an MS interface of 250° C., and ion source temperature of 200° C. The measurement mode was the scan or SIM mode; a scan range was 65-700 Da; and the sample was injected for 0.54 in a splitless mode.

Data Analysis of GC-MS

Peaks in measurement data of GCMS were waveform-processed using data analysis software GCMS solution and NIST 11 and GCMS metabolism component database (Shimadzu Corporation), and identification and quantification of detected metabolites were performed. A significant difference (p value) was calculated using the student's t-test.

In order to examine variations between samples of all detected peaks, peaks were extracted using Profiling Solution from data obtained by scan analysis; principal component analysis PCA, OPLS-DA and S-Plots analysis were performed using multivariable analysis software SIMCA ver.13.0.3 (Umetrics); and extraction of peaks that were varying in cells that had undergone EMT was performed.

Results

GCMS analysis of the metabolites that were prepared according to the above steps was performed. With respect to the HepG2 cells that were not stimulated by BMP-9 and the HepG2 cells that had undergone EMT, scan analyses of intracellularly and extracellularly secreted metabolites were performed. As a result, with respect to each of the total ion chromatogram (TIC) of the intracellular metabolites and the TIC of the extracellular metabolites, a large number of characteristic peaks were observed. As main peaks that were detected by the GCMS analysis, there were 130 peaks for the intracellular metabolites and 132 peaks for the extracellular metabolites (including medium components). Among the detected peaks, about 80 types of metabolites were detected to have a similarity of 75% or more to EI spectrum peaks of the NIST 11 and GCMS metabolism component database (Shimadzu Corporation). The metabolites of the cells that had undergone EMT and the HepG2 cells were measured in the scan and SIM modes and comparative quantification was performed. The results suggested that primary metabolites, that is, amino acids, which are produced when nutrients are converted into energy sources in the cells, change due to the EMT of the cells.

Among the metabolites that were detected from the extracts of the HepG2 cells that were stimulated with BMP-9 and had undergone EMT and the HepG2 cells that were not stimulated with BMP-9, main metabolites that could be identified were subjected to SIM (selective ion monitoring) analysis. The results are illustrated in the following tables. In the tables, the variations of metabolites of the HepG2 cells that had undergone EMT with respect to the metabolites of the HepG2 cells that were not stimulated were expressed in fold changes. Further, as significance, a symbol * represents a P value <0.05, and a symbol ** represents a P value <0.01.
a) Intracellular Metabolite List (SIM Analysis)

TABLE 1
Intracellular Metabolite List (SIM Analysis)

| Molecule | Fold | Signifi- | Molecule | Fold | Signifi- |
|---|---|---|---|---|---|
| Name | Change | cance | Name | Change | cance |
| Isoleucine | 0.79 | ** | Glutamic Acid | 1.42 | |
| Valine | 1.47 | ** | Phenylalanine | 1.87 | ** |
| Urea | 1.90 | | Asparagine | 0.84 | |
| Serine | 0.64 | ** | Acetylaspartic | 0.74 | * |
| | | | Acid | | |
| Leucine | 1.49 | ** | Glutamine | 0.56 | ** |
| Glycerol | 0.89 | | Citric Acid | 1.10 | |
| Alloisoleucine | 1.67 | ** | N-α-Acetyl-L- | 1.00 | |
| | | | Lysine | | |
| Proline | 1.92 | ** | D-(−)-tagatose | 0.62 | * |
| Glycine | 1.28 | * | d-glucose | 0.58 | ** |
| Fumaric Acid | 1.13 | | Lysine | 1.41 | |
| Threonine | 1.59 | ** | Tyrosine | 2.13 | ** |
| Alanine | 0.93 | | Tryptophan | 0.92 | |
| Amino Malonic | 1.04 | | β-D- | 0.67 | ** |
| Acid | | | Mannopyranoside | | |
| Malic Acid | 0.92 | | Myo-Inositol | 0.86 | |
| Methionine | 0.83 | | Cholesterol | 0.90 | |
| 5-Oxoproline | 1.86 | | | | |
| Aspartic Acid | 0.88 | ** | | | |
| 4- | 1.69 | ** | | | |
| Hydroxyproline | | | | | |
| Cysteine | 1.51 | ** | | | |
| Pentanedioic | 4.19 | | | | |
| Acid | | | | | |
| (Glutaric Acid) | | | | | |

As illustrated in Table 1, the results of the GC-MS analysis of the intracellular metabolites indicate that those that are significant in variation of amino acid are serine, aspartic acid, glutamine and isoleucine, which decreased in the cells that had undergone EMT. Further, branched-chain amino acids such as valine and leucine, essential amino acids such as threonine, phenylalanine and tyrosine, cysteine, glycine and proline increased in the cells that had undergone EMT. Further, an amount of intracellular glucose decreased along with EMT.

Further experiments on an increased number of samples were conducted, which showed that proline, threonine, cysteine, and tyrosine had lower correlation with epithelial-mesenchymal transition than the other metabolic substances.

b) Extracellular Metabolite List (SIM Analysis)

TABLE 2
Extracellular Metabolite List (SIM Analysis)

| Molecule | Fold | Signifi- | Molecule | Fold | Signifi- |
|---|---|---|---|---|---|
| Name | Change | cance | Name | Change | cance |
| Isoleucine | 1.37 | | Glutamic Acid | 1.12 | |
| Valine | 1.04 | | Phenylalanine | 1.74 | ** |
| Urea | 1.00 | | Acetylaspartic | 1.05 | |
| | | | Acid | | |
| Serine | 0.75 | ** | Asparagine | 0.41 | ** |
| Leucine | 1.04 | | Citric Acid | 1.69 | ** |
| Glycerol | 1.32 | * | N-α-Acetyl- | 1.11 | |
| | | | L-Lysine | | |
| Alloisoleucine | 1.01 | | Lysine | 1.41 | |
| L-Proline | 1.22 | * | Tyrosine | 1.06 | |
| Glycine | 1.01 | | Myo-Inositol | 1.01 | |
| Fumaric Acid | 2.06 | ** | Tryptophan | 0.51 | * |
| Threonine | 0.99 | | Inositol | 0.69 | |
| Alanine | 1.05 | | Cholesterol | 0.82 | * |
| Amino Malonic | 0.75 | ** | | | |
| Acid | | | | | |
| Malic Acid | 1.97 | ** | | | |
| Methionine | 0.82 | ** | | | |
| 5-Oxoproline | 1.09 | | | | |
| Aspartic Acid | 0.53 | ** | | | |
| 4-Hydroxyproline | 0.99 | | | | |
| Cysteine | 0.96 | | | | |
| Pentanedioic Acid | 0.99 | | | | |
| (Glutaric Acid) | | | | | |

As illustrated in Table 2, the results of the GC-MS analysis of the extracellular metabolites indicate that amino acids such as serine, asparagine, aspartic acid, methionine and tryptophan significantly decreased in the cells that had undergone EMT, and L-proline and phenylalanine significantly increased. Further, fumaric acid, malic acid and citric acid, which are intermediate metabolites in a TCA cycle, significantly increased in the cells that had undergone EMT.

As described above, by analyzing metabolism variations of cells that had undergone epithelial-mesenchymal transition, as illustrated in Tables 1 and 2, statistically significant changes were observed for various metabolic substances. In particular, with respect to serine, phenylalanine and aspartic acid, significant changes were observed in both intracellular metabolites and extracellular metabolites, and these may be used, each alone or in combination, as indicators for determining whether or not cells have undergone epithelial-mesenchymal transition. However, indicators provided by the present invention are not limited to these. Other metabolic substances that have shown statistical significance, specifically, isoleucine, valine, leucine, alloisoleucine, proline, threonine, 4-hydroxyproline, cysteine, glutamine, d-glucose, tyrosine, β-D-mannopyranoside, glycine, acetylaspartic acid, D-(−)-tagatose, fumaric acid, amino malonic acid, malic acid, methionine, asparagine, citric acid, glycerol, L-proline, tryptophan and cholesterol, may also be used, each alone or in combination, as indicators for determining whether or not cells have undergone epithelial-mesenchymal transition.

The technologies described in the present specification are based on the present inventors' findings that there is a relationship between epithelial-mesenchymal transition and a variation in a metabolite level, and that variation in a metabolite level indicates characteristics of stem cells.

The present inventors have performed identification of varying metabolites during EMT of liver cancer cells by performing metabolome analysis using GC-MS that ensures the most complete coverage among the omics analyses. Based on findings about identified metabolic substances, the present invention provides, in one aspect, a method for determining whether or not the cells have undergone EMT. Further, the present invention provides, in another aspect, a method in which, by analyzing metabolic substances of certain cells, whether or not the cells have undergone epithelial-mesenchymal transition is determined and this information is used to screen drug candidate compounds.

That is, the present invention includes the following aspects.

[1] A method for determining whether or not cells have undergone epithelial-mesenchymal transition, includes: i) a step of measuring serine in a metabolic substance mixture prepared from culture of the cells; and ii) a step of comparing the measurement result with a reference.

[2] A method for analyzing whether or not cells that have undergone epithelial-mesenchymal transition are detected from a tissue sample, includes: i) a step of measuring serine in a metabolic substance mixture prepared from the tissue sample; and ii) a step of comparing the measurement result with a reference.

[3] The method described in any one of [1] and [2] further includes a step of measuring phenylalanine in the metabolic substance mixture and comparing the measurement result with a reference.

[4] The method described in any one of [1]-[3] further includes a step of measuring aspartic acid in the metabolic substance mixture and comparing the measurement result with a reference.

[5] The method described in any one of [1]-[4] further includes a step of measuring one or more metabolic substances selected from the group consisting of isoleucine, valine, leucine, alloisoleucine, proline, threonine, 4-hydroxyproline, cysteine, glutamine, d-glucose, tyrosine, β-D-mannopyranoside, glycine, acetylaspartic acid, D-(−)-tagatose, fumaric acid, amino malonic acid, malic acid, methionine, asparagine, citric acid, glycerol, L-proline, tryptophan and cholesterol in the metabolic substance mixture, and comparing the measurement result with a reference.

[6] The method described in any one of [1]-[5] further includes a step of determining whether or not cells that have undergone epithelial-mesenchymal transition are cancer stem cells, by examining one or more genetic markers selected from the group consisting of CD19, CD20, CD24 (HSA), CD38, CD44 (PGP1), CD90 (THY1), CD133 (prominin 1), EpCAM (epithelial cell adhesion molecule), E-cadherin and ATP-binding cassette B5 (ABCB5), with respect to the cells that have undergone epithelial-mesenchymal transition.

[7] A method for screening a drug candidate compound includes:

(a) a step of culturing cells under a condition in which cancer cells and a drug candidate compound are in contact with each other,
(b) when growth of the cancer cells is inhibited or the number of the cancer cells is reduced under the culture condition, with respect to the cells of which the growth is inhibited or the cells of which the number is reduced, a step of preparing a metabolic substance mixture from cell culture obtained by the step (a) and measuring serine in the metabolic substance mixture; and
(c) a step of comparing the measurement result with a reference, and
when cells that have undergone epithelial-mesenchymal transition are not detected from the cell culture by comparing with the reference, it is determined that the drug candidate compound affects the cells that have undergone epithelial-mesenchymal transition in the cancer cells.

[8] The method described in [8] further includes a step of measuring phenylalanine in the metabolic substance mixture and comparing the measurement result with a reference.

[9] The method described in any one of [7] and [8] further includes a step of measuring aspartic acid in the metabolic substance mixture and comparing the measurement result with a reference.

[10] In the method described in any one of [1]-[9], measurements of the metabolic substances are performed using gas chromatography-mass spectrometry (GCMS), liquid chromatography-mass spectrometry (LC-MS), capillary electrophoresis analysis-mass spectrometry (CE-MS), Fourier transform ion cyclotron resonance mass spectrometry (FT-ICR-MS) or high-performance liquid chromatography-mass spectrometry (HPLC-MS).

The results of the metabolome analysis revealed that metabolite profiles change along with EMT in the liver cancer cells. Further, a large number of varying metabolic substances have been identified. Based on such findings of the metabolism variation, whether or not the cells are cells that have undergone EMT can be analyzed. Further, by analyzing variations of a plurality of metabolic substances, sensitivity and specificity of the analysis can be improved. Further, whether or not cells that have undergone epithelial-mesenchymal transition are detected from a certain tissue can be analyzed. When cells that have undergone epithelial-mesenchymal transition are detected, it is further possible to examine the metastatic potential of the cells and to analyze whether or not the cells are cancer stem cells by using in conjunction with other cancer stem cell markers. As a result, it is possible to evaluate results of cancer treatment, to determine prognosis, and to determine whether or not additional metastatic cancer treatment regimen is necessary. Further, effects of the metastatic cancer treatment can be monitored and evaluated.

### INDUSTRIAL APPLICABILITY

In the method according to one aspect of the present invention, whether or not cells have undergone epithelial-mesenchymal transition or whether or not cells that have undergone epithelial-mesenchymal transition are contained in a tissue can be determined. Further, by using additional genetic markers and the like, it is also possible to determine whether or not cancer cells that have undergone epithelial-mesenchymal transition are cancer stem cells. As a result, it is possible to evaluate results of cancer treatment, to screen drug candidate compounds, and to determine whether or not metastatic cancer treatment regimen is necessary. Further, effects of the metastatic cancer treatments can be evaluated.

The entire contents of all publications, patents and patent applications that are cited in the present specification are incorporated by reference in the present specification.

Obviously, numerous modifications and variations of the present invention are possible in light of the above teachings. It is therefore to be understood that within the scope of the appended claims, the invention may be practiced otherwise than as specifically described herein.

What is claimed is:

1. A method for detecting epithelial-mesenchymal transition of a cell, comprising:
measuring a level of serine in a metabolic substance mixture prepared from culture of a cell; and
comparing the level of serine with a reference for serine such that epithelial-mesenchymal transition of the cell is detected based on at least the level of serine compared with the reference.

2. The method of claim 1, wherein epithelial-mesenchymal transition of the cell is detected when the level of serine is lower than the reference.

3. The method of claim 2, further comprising:
measuring a level of phenylalanine in the metabolic substance mixture; and
comparing the level of phenylalanine with a reference for phenylalanine such that epithelial-mesenchymal transition of the cell is detected based on at least the level of serine and the level of phenylalanine compared with the references, respectively.

4. The method of claim 3, wherein epithelial-mesenchymal transition of the cell is detected when the level of serine is lower than the reference for serine and the level of phenylalanine is higher than the reference for phenylalanine.

5. The method of claim 1, further comprising:
measuring a level of aspartic acid in the metabolic substance mixture; and
comparing the level of aspartic acid with a reference for aspartic acid such that epithelial-mesenchymal transition of the cell is detected based on at least the level of serine and the level of aspartic acid compared with the references, respectively.

6. The method of claim 5, wherein epithelial-mesenchymal transition of the cell is detected when the level of serine is lower than the reference for serine and the level of aspartic acid is lower than the reference for aspartic acid.

7. The method of claim 1, further comprising:
measuring a level of at least one substance selected from the group consisting of isoleucine, valine, leucine, alloisoleucine, 4-hydroxyproline, glutamine, d-glucose, β-D-mannopyranoside, glycine, acetylaspartic acid, D-(−)-tagatose, fumaric acid, amino malonic acid, malic acid, methionine, asparagine, citric acid, glycerol, L-proline, tryptophan and cholesterol, in the metabolic substance mixture; and
comparing the level of the substance with a reference for the substance such that epithelial-mesenchymal transition of the cell is detected based on at least the level of serine and the level of the substance compared with the references, respectively.

8. The method of claim 1, further comprising:
analyzing at least one genetic marker selected from the group consisting of CD19, CD20, CD24, CD38, CD44, CD90, CD133, EpCAM, E-cadherin and ATP-binding cassette B5, with respect to the cell where epithelial-mesenchymal transition is detected, to determine whether the cell is a cancer stem cell.

9. The method of claim 1, wherein the measuring of the level of serine is performed by one of gas chromatography-mass spectrometry, liquid chromatography-mass spectrometry, capillary electrophoresis analysis-mass spectrometry, Fourier transform ion cyclotron resonance mass spectrometry, and high-performance liquid chromatography-mass spectrometry.

10. A method for detecting epithelial-mesenchymal transition of a cell, comprising:
measuring a level of serine in a metabolic substance mixture prepared from a tissue sample; and
comparing the level of serine with a reference for serine such that epithelial-mesenchymal transition of the cell is detected based on at least the level of serine compared with the reference.

11. The method of claim 10, wherein epithelial-mesenchymal transition of the cell is detected when the level of serine is lower than the reference.

12. The method of claim 11, further comprising:
measuring a level of phenylalanine in the metabolic substance mixture; and
comparing the level of phenylalanine with a reference for phenylalanine such that epithelial-mesenchymal transition of the cell is detected based on at least the level of serine and the level of phenylalanine compared with the references, respectively.

13. The method of claim 12, wherein epithelial-mesenchymal transition of the cell is detected when the level of serine is lower than the reference for serine and the level of phenylalanine is higher than the reference for phenylalanine.

14. The method of claim 10, further comprising:
measuring a level of aspartic acid in the metabolic substance mixture; and
comparing the level of aspartic acid with a reference for aspartic acid such that epithelial-mesenchymal transition of the cell is detected based on at least the level of serine and the level of aspartic acid compared with the references, respectively.

15. The method of claim 14, wherein epithelial-mesenchymal transition is detected of the cell when the level of serine is lower than the reference for serine and the level of aspartic acid is lower than the reference for aspartic acid.

16. The method of claim 10, further comprising:
measuring a level of at least one substance selected from the group consisting of isoleucine, valine, leucine, alloisoleucine, 4-hydroxyproline, glutamine, d-glucose, β-D-mannopyranoside, glycine, acetylaspartic acid, D-(−)-tagatose, fumaric acid, amino malonic acid, malic acid, methionine, asparagine, citric acid, glycerol, L-proline, tryptophan and cholesterol, in the metabolic substance mixture; and
comparing the level of the substance with a reference for the substance such that epithelial-mesenchymal transition of the cell is detected based on at least the level of serine and the level of the substance compared with the references, respectively.

17. The method of claim 10, wherein the measuring of the level of serine is performed by one of gas chromatography-mass spectrometry, liquid chromatography-mass spectrometry, capillary electrophoresis analysis-mass spectrometry, Fourier transform ion cyclotron resonance mass spectrometry, and high-performance liquid chromatography-mass spectrometry.

18. A method for detecting epithelial-mesenchymal transition of a cancer cell, comprising:
measuring a level of serine in a metabolic substance mixture prepared from culture of a cancer cell taken from a subject; and
comparing the level of serine with a previous level of serine measured for the subject such that epithelial-mesenchymal transition of the cancer cell is detected based on at least the level of serine compared with the previous level of serine.

19. The method of claim 18, wherein epithelial-mesenchymal transition of the cancer cell is detected when the level of serine is lower than the previous level of serine.

20. The method of claim 19, further comprising:
measuring a level of phenylalanine in the metabolic substance mixture; and
comparing the level of phenylalanine with a previous level for phenylalanine such that epithelial-mesenchymal transition of the cancer cell is detected based on at least the level of serine and the level of phenylalanine compared with the previous levels, respectively.

21. The method of claim 20, wherein epithelial-mesenchymal transition of the cancer cell is detected when the level of serine is lower than the previous level for serine and the level of phenylalanine is higher than the previous level for phenylalanine.

22. The method of claim 10, further comprising:
measuring a level of aspartic acid in the metabolic substance mixture; and
comparing the level of aspartic acid with a previous level for aspartic acid such that epithelial-mesenchymal transition of the cancer cell is detected based on at least the level of serine and the level of aspartic acid compared with the previous levels, respectively.

23. The method of claim 22, wherein epithelial-mesenchymal transition of the cancer cell is detected when the level of serine is lower than the previous level for serine and the level of aspartic acid is lower than the previous level for aspartic acid.

24. A method for screening a drug candidate compound, comprising:
culturing a cancer cell and a drug candidate compound;
preparing a metabolic substance mixture from the culturing of the cancer cell whose cell growth is inhibited or cell number is reduced;
measuring a level of serine in the metabolic substance mixture; and
comparing the level of serine with a reference for serine to determine whether epithelial-mesenchymal transition of the cancer cell has occurred based on at least the level of serine compared with the reference.

25. The method of claim 24, wherein epithelial-mesenchymal transition of the cancer cell is detected when the level of serine is lower than the reference.

26. The method of claim 24, further comprising:
measuring a level of phenylalanine in the metabolic substance mixture; and
comparing the level of phenylalanine with a reference for phenylalanine to determine whether epithelial-mesenchymal transition of the cancer cell has occurred based on at least the level of serine and the level of phenylalanine compared with the references, respectively.

27. The method of claim 26, wherein epithelial-mesenchymal transition of the cancer cell is detected when the level of serine is lower than the reference for serine and the level of phenylalanine is higher than the reference for phenylalanine.

28. The method of claim 27, further comprising:
measuring a level of aspartic acid in the metabolic substance mixture; and
comparing the level of aspartic acid with a reference for aspartic acid to determine whether epithelial-mesenchymal transition of the cancer cell has occurred based on at least the level of serine and the level of aspartic acid compared with the references, respectively.

29. The method of claim 28, wherein epithelial-mesenchymal transition of the cancer cell is detected when the level of serine is lower than the reference for serine and the level of aspartic acid is lower than the reference for aspartic acid.

30. A method for assessing efficacy of a treatment in a subject suffering from a cancer, comprising:
detecting epithelial-mesenchymal transition of a cancer cell in a subject by the method of claim 18.
